(19) United States
(12) Patent Application Publication
(10) Pub. No.: US 2025/0231161 A1
(43) Pub. Date: Jul. 17, 2025

(54) PRESSURE-VOLUME-TEMPERATURE (PVT) SYSTEM FOR RESERVOIR FLUID CHARACTERIZATION

(71) Applicant: SAUDI ARABIAN OIL COMPANY, Dhahran (SA)

(72) Inventors: Khalid M. Alhajri, Alkhobar (SA); Omar M. Hassan, Dammam (SA); Fawaz M. Alotaibi, Dammam (SA)

(73) Assignee: SAUDI ARABIAN OIL COMPANY, Dhahran (SA)

(21) Appl. No.: 18/414,228

(22) Filed: Jan. 16, 2024

(51) Int. Cl. G01N 33/00; E21B 49/08; G01N 33/28

(52) U.S. Cl. CPC G01N 33/0062; G01N 33/28; E21B 49/08; E21B 2200/20; G01N 33/0068

(57) ABSTRACT

A pressure-volume-temperature (PVT) system for characterization of a reservoir fluid, includes: a temperature control unit; a sample cell disposed inside the temperature control unit for accommodating the reservoir fluid, the temperature control unit includes a housing and an interior chamber defined by a space between the sample cell and the housing; and a fluid supply unit that provides a temperature control fluid to the interior chamber to control a temperature of the sample cell. The fluid supply unit includes a first container providing a first fluid having a first temperature as the temperature control fluid for a first stage of PVT analysis and a second fluid having a second temperature that is different from the first fluid as the temperature control fluid for a second stage for PVT analysis.

### BACKGROUND

Oil and gas extraction from subsurface rock formations requires drilling of one or more wells using drilling rigs mounted on surface ground or on offshore rig platforms. Once drilled, the wells may access a reservoir fluid, which is a mixture contained within the reservoir where hydrocarbons of interest are accumulated in the porous or fractured rock formations. The reservoir fluid may include liquid hydrocarbons, aqueous solutions with dissolved salts, hydrocarbon gases such as methane, and non-hydrocarbon gases such as hydrogen sulfide and carbon dioxide.

Characterization of the reservoir fluid plays a key role in different upstream and downstream applications. The characterization data may be used to instruct one or more oilfield applications including formation and reservoir quality assessments, reservoir behavior forecasting, reservoir modeling and simulation, wellbore planning, designing and optimization of injection and production strategies, and efficient reservoir management.

Various strategies may be applied to characterize the reservoir fluid, such as phase behavior analysis using a pressure-volume-temperature (PVT) system. Phase behavior analysis may include fluid analysis used in predicting the fluid's behavior under different pressures (P), volumes (V), and temperatures (T). Accordingly, phase behavior analysis may also be referred to as PVT analysis.

PVT analysis is often performed on a reservoir fluid as soon after the reservoir fluid is collected as possible. However, conventional PVT systems require an oven to control the temperature, which occupies a majority of a volume of the PVT system and requires a long period of time before the PVT system reaches an equilibrium temperature for PVT analysis. Accordingly, for better evaluating, designing, or modeling oil and gas extraction and processing systems, there exists a need to improve the quality and efficiency of characterization strategies with reduced size, low cost, and high efficiency.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In some aspects, the techniques described herein relate to a pressure-volume-temperature (PVT) system for characterization of a reservoir fluid, including: a temperature control unit; a sample cell disposed inside the temperature control unit for accommodating the reservoir fluid, wherein the temperature control unit includes a housing and an interior chamber defined by a space between the sample cell and the housing; and a fluid supply unit that provides a temperature control fluid to the interior chamber to control a temperature of the sample cell; wherein the fluid supply unit contains a first container providing a first fluid having a first temperature as the temperature control fluid for a first stage of PVT analysis and a second fluid having a second temperature that is different from the first fluid as the temperature control fluid for a second stage for PVT analysis.

In some aspects, the techniques described herein relate to a PVT system, further including a piston arranged in the sample cell and configured to control a pressure inside the sample cell.

In some aspects, the techniques described herein relate to a PVT system, wherein the first container is configured to preheat the first fluid to the first temperature before the first stage and preheat the second fluid to the second temperature before the second stage.

In some aspects, the techniques described herein relate to a PVT system, wherein the second fluid replaces the first fluid in the interior chamber after the first stage, such that a temperature in the interior chamber changes from the first temperature to the second temperature after replacement.

In some aspects, the techniques described herein relate to a PVT system, wherein the reservoir fluid is characterized in the sample cell under steady state conditions to obtain a property of the reservoir fluid.

In some aspects, the techniques described herein relate to a PVT system, further including a controller that generates a reservoir model based on the property of the reservoir fluid.

In some aspects, the techniques described herein relate to a method for performing a pressure-volume-temperature (PVT) analysis, including: collecting a reservoir fluid from a reservoir; performing PVT analysis on the reservoir fluid using a sample cell disposed in a temperature control unit; controlling a temperature of the sample cell using a temperature control fluid in an interior chamber defined by a space between the sample cell and a housing of the temperature control unit, providing, for a first stage of the PVT analysis, a first fluid having a first temperature as the temperature control fluid, and providing, for a second stage of the PVT analysis, a second fluid having a second temperature that is different from the first temperature as the temperature control fluid.

In some aspects, the techniques described herein relate to a method, further including controlling a pressure inside the sample cell by a piston arranged in the sample cell.

In some aspects, the techniques described herein relate to a method, further including preheating the first fluid in a first container to the first temperature before the first stage, and preheating the second fluid in a second container to the second temperature before the second stage.

In some aspects, the techniques described herein relate to a method, further including replacing the first fluid in the interior chamber with the second fluid after the first stage, such that a temperature in the interior chamber changes from the first temperature to the second temperature after replacement.

In some aspects, the techniques described herein relate to a method, further including determining a property of the reservoir fluid by characterizing the reservoir fluid in the sample cell under steady state conditions.

In some aspects, the techniques described herein relate to a method, further including generating a reservoir model based on the property of the reservoir fluid.

Other aspects and advantages of the claimed subject matter will be apparent from the following description and the appended claims.

### BRIEF DESCRIPTION OF DRAWINGS

FIG. 1 depicts a pressure-volume-temperature (PVT) system in accordance with one or more embodiments.

FIG. 2 depicts a flowchart in accordance with one or more embodiments.

FIG. 3 depicts a reservoir simulator in accordance with one or more embodiments.

FIG. 4 depicts a system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural references unless the context clearly dictates otherwise. Thus, for example, reference to a “fluid sample” includes reference to one or more of such fluid samples.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowchart may be omitted, repeated, and/or performed in a different order than the order shown. Accordingly, the scope disclosed herein should not be considered limited to the specific arrangement of steps shown in the flowchart.

Although multiple dependent claims are not introduced, it would be apparent to one of ordinary skill that the subject matter of the dependent claims of one or more embodiments may be combined with other dependent claims.

In one aspect, embodiments disclosed herein generally relate to characterization of a fluid from a reservoir using phase behavior analysis (referred to herein as PVT analysis). The PVT analysis may be conducted on the reservoir fluid to simulate what takes place in the reservoir and/or on the surface during reservoir production. Accordingly, PVT analysis may be conducted, at least in part, under pressures and temperatures that mimic the pressures and temperatures in which the reservoir fluid is exposed to in the reservoir and/or during production. PVT analysis may include separating phases from the reservoir fluid and analysis of the reservoir fluid as it undergoes phase separation. For example, PVT analysis may include measuring properties of a reservoir fluid, such as phase behavior, heat capacity, density, viscosity, and compressibility. Data obtained from PVT analysis may be used for evaluating formations and reservoir, for setting parameters in reservoir modeling and simulation, for wellbore planning in enhanced oil recovery processes, for designing injection and production systems, and for other reservoir production or management processes.

PVT analysis is often performed on a reservoir fluid as soon after the reservoir fluid is collected as possible. For example, reservoir fluid samples are often collected while drilling a first exploration well into the reservoir, before the reservoir is put into full production. Once a reservoir fluid sample is collected, it may be transferred to a PVT system described in the present disclosure, where it may be tested at certain pressures and temperatures, in a time-efficient manner, to determine phase behavior and study compositional changes of the reservoir fluid through the life of the reservoir as pressure decreases.

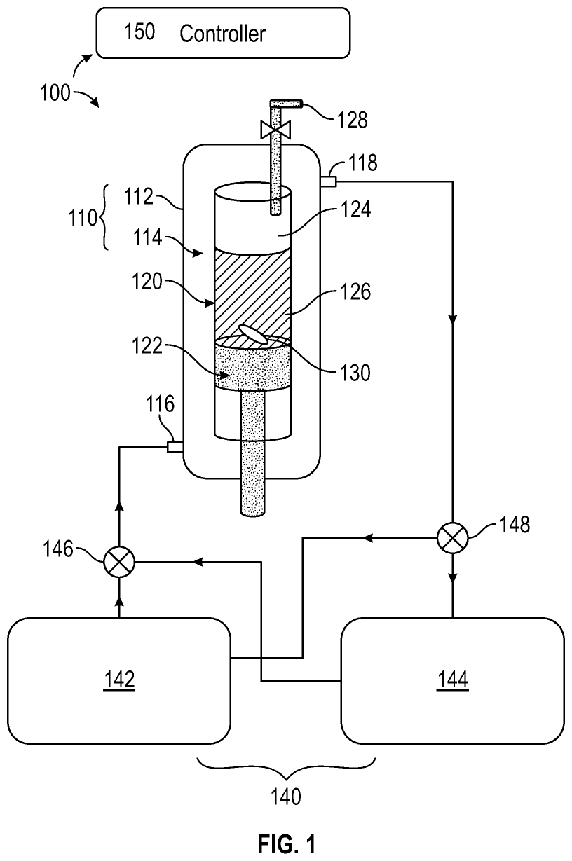

FIG. 1 shows a PVT system 100 according to one or more embodiments of the present disclosure. The PVT system 100 includes a temperature control unit 110, a sample cell 120 disposed inside the temperature control unit 110, a fluid supply unit 140, and a controller 150. The temperature control unit 110 is configured to provide a thermally isolated environment for the sample cell 120 and accommodate a temperature control fluid that heats or cools the sample cell 120, as well as any contents within the sample cell. The sample cell 120 is configured to accommodate a sample, for example a reservoir fluid, for PVT analysis. The fluid supply unit 140 is configured to provide the temperature control fluid in a controlled manner. The controller 150 is configured to control one or more operation parameters for components in the PVT system, as well as receive, transmit, process, store, or manage data and information associated with the PVT system.

The temperature control unit 110 has a housing 112 and an interior chamber 114 defined by a space between the housing and an outer periphery of the sample cell 120. The housing 112 may be made of, or coated with, an insulating material, providing a thermally stable environment to the interior chamber and the sample cell. The insulating material may be any known material that reduces heat loss and protect workforce from potential burns, such as, elastomeric foam (e.g., polyurethane foam), silicone, fiberglass, and mineral wool, The housing 112 may have an inlet 116 that receives a temperature control fluid into the interior chamber 114 and an outlet 118 that discharges the temperature control fluid out of the interior chamber 114. In one or more embodiments, the inlet 116 is arranged on a side surface of the housing near a bottom surface of the housing, and the outlet 118 is arranged on a side surface of the housing near a top surface of the housing. The temperature control fluid is used to control a temperature of the sample cell 120. A temperature inside the sample cell, including any contained fluid, is considered to be substantially the same as the temperature of the interior chamber, or the same as the temperature of the temperature control fluid under steady state conditions, when conditions for the PVT analysis may be kept constant over at least a period of time during the PVT analysis, and when the temperature control fluid is not flowing.

In one or more embodiments, the temperature control fluid may be obtained from a fluid supply unit 140. The fluid supply unit 140 is configured to supply at least one liquid having a certain temperature to the interior chamber 114 as the temperature control fluid. In one or more embodiments, the PVT analysis may include a plurality of stages conducted under different temperatures. One or more fluids may be supplied to obtain the temperature control fluid at each of the stages. As shown in a non-limiting example of FIG. 1, the fluid supply unit 140 may include a first container 142 holding a first fluid having a first temperature and a second container 144 holding a second fluid having a second temperature that is different from the first temperature. A controlled amount of the first fluid and/or second fluid may be supplied using a first flow controller 146 as the temperature control fluid. The temperature control fluid may be subsequently introduced to the interior chamber 114 of the temperature control unit 110 through the inlet 116, so as to control (increase or decrease) the temperature of the sample cell 120. In one or more embodiments, the first fluid is supplied to the interior chamber 114 as the temperature control fluid for a first stage during PVT analysis, and the second fluid is supplied as the temperature control fluid for a second stage which requires a different temperature from the first stage. The temperature control fluid may be discharged from the interior chamber through the outlet 118, for example after each stage or after the PVT analysis, and subsequently recycled back to the containers using the second flow controller 148. The first fluid and the second fluid may have substantially the same composition and may be composed of any common heat transfer fluid, for example, mineral oil or synthetic hydrocarbon- or silicone-based fluids.

The PVT system according to one or more embodiments described herein provides a configuration ensuring that the temperature control fluid having the desired temperature is obtained immediately for stages of the PVT analysis requiring different temperatures, which greatly reduces a time of temperature control in conventional PVT systems using, for example, an oven. Further, without the use of an oven, the PVT system disclosed herein occupies a much smaller space compared to conventional PVT systems.

Although not shown in the figures, one having ordinary skill in that art would readily recognize that the PVT system in the present disclosure may further comprise one or more valves, piping or tubing and respective connections, and flow control devices (e.g., flowmeter), which may be disposed at any position that a fluid may present, for example, at the inlet 116 and outlet 118 of the temperature control unit 110, at the sample cell 120 where the reservoir fluid to be analyzed is introduced, at the flow controllers 146, 148, in the containers 142, 144, or along pipes or tubings connecting the flow controllers, the containers, and other components of the PVT system. In some embodiments, the valves and pipings or tubings may be made of or coated with a material that withstands corrosion, high temperature, and high pressure. Likewise, valve connections may be made of a non-corrosive, high-pressure high-temperature material. In some embodiments, to prevent thermal loss, the valves and pipings or tubings may be insulated.

In one or more embodiments, the PVT system 100 includes a temperature sensor (e.g., thermocouple) and a temperature transmitter (not shown), where the temperature transmitter can transmit one or more temperatures as sensed by the temperature sensor, such as the temperature of the interior chamber, the temperature of the temperature control fluid, and the temperature inside the sample cell, to an external device and/or a control device. The temperature sensor may be disposed along a flow path of the temperature control fluid before entering the temperature control unit 110, inside the housing 112 and outside the sample cell 120, or may be disposed within the sample cell 120. In some instances, the temperature sensor and the temperature transmitter may be integrated in a same device. That is, one or more devices described herein may be capable of both sensing and transmitting functionalities.

In one or more embodiments, the sample cell 120 is configured to accommodate a sample of interest, such as a reservoir fluid, during PVT analysis. The reservoir fluid may be acquired from a wellbore, a fracture in a formation, a body of water or oil or mixture of materials, or other void in a subterranean formation that is large enough from which to collect a sample. Without loss of generality, the reservoir fluid may contain solid particles such as sand, salt crystals, proppant, solid acids, solid or viscous hydrocarbon, viscosity modifiers, weighing agents, completions residue, or drilling debris. Further, the reservoir fluid may contain water (for example, salt water), hydrocarbons, drilling mud, emulsions, fracturing fluid, and other chemical constituents often used in drilling operations such as viscosifiers, surfactants, and/or dissolved gases. In one or more embodiments, the PVT system further includes a high-pressure filter (not shown), such that the reservoir fluid is filtered to remove particulates before introduced to the sample cell 120.

A pressure within the sample cell may be changed (increased or reduced) such that the reservoir fluid is subjected to different pressures and undergoes different phase changes during the PVT analysis. The pressure may be controlled by various devices known in the art. For example, in the non-limiting example shown in FIG. 1, movement of a piston 122 may be used to change the pressure within the sample cell. The piston 122 may be motorized by a motor (not shown) to change the pressure within the sample cell in a range of more than 0 to about 10,000 psi. In other embodiments, a pump (not shown) may be used to pressurize or evacuate (create a vacuum within) the sample cell.

When the pressure within the fluid sample chamber in the sample cell 120 is changed (reduced or increased), the reservoir fluid inside the sample cell may separate into a gas phase 124 and a liquid phase 126. The gas phase may be discharged from the sample cell through a discharge outlet 128 of the sample cell and may be controlled by one or more valves.

In one or more embodiments, the sample cell 120 may be made of a material capable of withstanding high pressure and high temperature. In addition, at least an interior surface of the sample cell may be made of or coated with a material capable of withstanding corrosive environments, such that sour gas conditions (e.g., carbon dioxide (CO₂) and hydrogen sulfide (H₂S)) would not cause damages to the sample cell. For example, the sample cell may be made of alloys, such as Hastelloy alloys.

In one or more embodiments, the PVT system further includes a mixer 130 disposed in the sample cell 120. The mixer 130 may be configured to bring turbulence to the sample inside the sample cell 120, such that a temperature distribution of the sample is uniform.

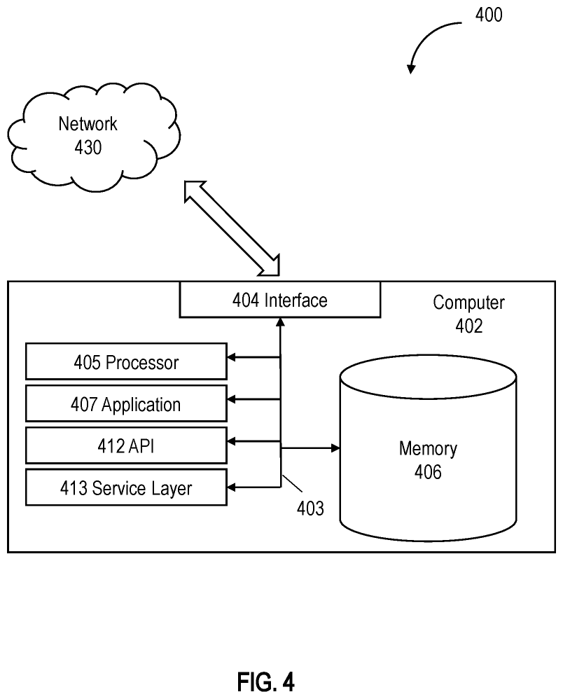

In one or more embodiments, the PVT system 100 further includes a controller 150. In one or more embodiments, the controller 150 can receive, process, and record signals generated by one or more components of the PVT system 100, such as the temperature control unit, the sample cell, the flow controllers, piston, and other devices that may present in the PVT system. Further, in one or more embodiments, the controller 150 can transmit control signals to alter the operation of the one or more components of the PVT system 100. In general, the connection of the controller 150 to components of the PVT system 100 may be wired or wireless. In one or more embodiments, the controller 150 is further connected to, and capable of receiving and transmitting signals to, one or more valves or any piping or tubing that connects components of the PVT system 100. In these embodiments, the controller 150 can identify and control the state (e.g., open or closed) of the valves of the PVT system. In one or more embodiments, the controller 150 may be used in processing data obtained from the PVT system, for example, processing a property value of the reservoir fluid to generate reservoir models or to set parameters in oilfield applications. The controller 150 may include one or more controllers and/or edge computing devices. In one or more embodiments, the controller may be implemented in a computing system, such as the computing system discussed below with reference to FIG. 4.

The PVT system according to one or more embodiments described in the present disclosure is used for characterization of a reservoir fluid, and the resulting characterization data may be used to guide oilfield applications such as drilling operation, reservoir modeling and simulation, wellbore planning, or production. In one or more embodiments, the PVT system may be used for analysis of a reservoir fluid as it undergoes phase changes during pressure changes between pressures above a bubble point of the fluid sample and pressures below the bubble point of the fluid sample. The bubble point refers to the pressure conditions at which the first bubble of gas comes out of solution in oil under a given temperature. When a reservoir is under a pressure above the bubble point (an undersaturated reservoir) prior to beginning production, the pressure may decrease as fluid is produced from the reservoir to a pressure below the bubble point, in which case, a gas phase may begin to form in the reservoir.

In one or more embodiments, the PVT system may be used in, for example, determining a formation volume factor (a ratio of a volume occupied by a fluid phase at reservoir conditions divided by a volume occupied by the fluid phase at surface conditions), a solution gas/oil ratio (a total standard volume of gas separated from the produced oil at atmospheric pressure, divided by a volume of the residual oil at standard conditions), a gas formation volume factor (gas volume at the formation pressure divided by the volume of the same gas at standard conditions), a compressibility factor for the separated gas phase (a correction factor describing the deviation of a real gas from ideal gas behavior), gas gravity of the separated gas phase (molecular weight of the gas divided by the molecular weight of atmospheric air), and others.

While only one configuration is shown in FIG. 1, one having ordinary skill in the art would recognize that the present disclosure is not intended to be limiting and other implementations may be used without departing from the scope of the disclosure. For example, a number of containers may be adjusted if the PVT analysis includes more stages requiring different temperatures, each fluid in the container may be used for a plurality of non-continuous stages of the PVT analysis, the temperature control fluid may be obtained by mixing two or more fluids from the fluid supply unit, a size and shape of the sample shell may be modified, the first fluid and the second fluid may have different compositions, etc.

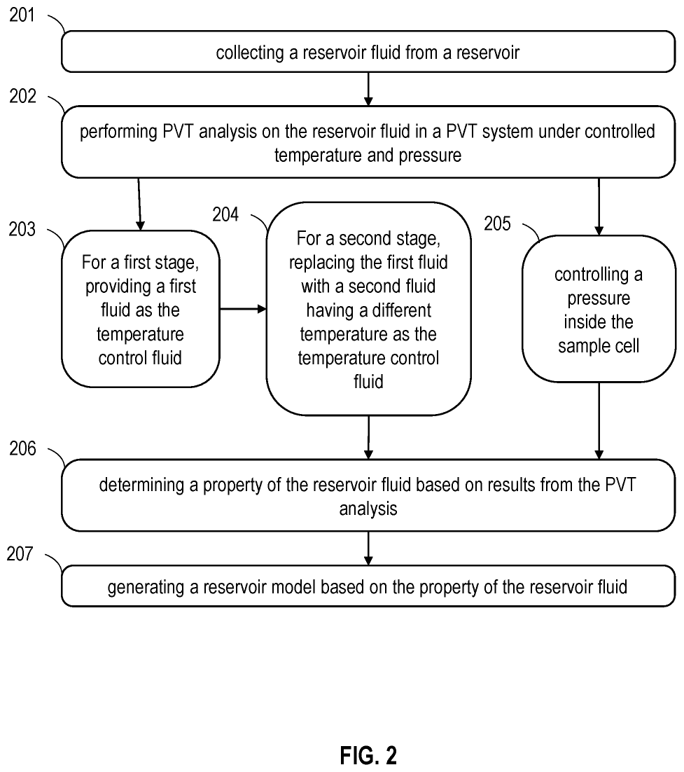

According to one or more embodiments of the present disclosure, methods for characterization of a reservoir fluid may include conducting a PVT analysis on the reservoir fluid from a well using a PVT system as described herein (e.g., a PVT system having a sample cell, which may contain the reservoir fluid, a temperature control unit, and fluid containers for immediate generation of a temperature control fluid), and applying the resulted data to instruct one or more oilfield applications, such as formation and reservoir quality assessments, reservoir behavior forecasting, reservoir modeling and simulation, wellbore planning, designing and optimization of injection and production strategies, and efficient reservoir management. Examples of methods according to one or more embodiments of the preset disclosure are described below with reference to FIG. 2.

FIG. 2 shows a schematic diagram of a method according to one or more embodiments of the present disclosure. While the various blocks in FIG. 2 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

The method may include a step 201 of collecting a reservoir fluid from a reservoir accessed by a well. The reservoir fluid may be collected at an early stage in the well's producing life, for example, from an exploratory well, to represent the reservoir under its initial conditions. The collecting of the reservoir fluid may be performed, for example, using downhole sampling, wellhead sampling, or surface recombination sampling processes and apparatuses.

In one or more embodiments, downhole sampling may include using a bottomhole sampler run downhole on a wireline to collect a reservoir fluid from a downhole location, for example a subsurface well stream, under bottomhole pressure. A bottomhole sampler may include a sample chamber and one or more fluidly connected valves that, when opened, collect a volume of the reservoir fluid to be stored in the sample chamber. In one or more embodiments, wellhead sampling may be used to collect the reservoir fluid, for example, when the reservoir fluid is an undersaturated oil. Such techniques may include collecting fluid from the wellhead (e.g., through a wellhead valve) and transferring the collected fluid directly to a PVT system (e.g., through tubing between the wellhead and the PVT system). Once a reservoir fluid is collected, it may be brought to the surface and transferred to a PVT system (e.g., PVT system 100) as soon as possible.

In one or more embodiments, the method includes a step 202 of performing PVT analysis on the reservoir fluid in a PVT system under controlled temperature and pressure. Specifically, once the reservoir fluid is collected, the reservoir fluid may be immediately transferred to a PVT system described in one or more embodiments of the present disclosure, where the reservoir fluid may be loaded into a sample cell for PVT analysis. The PVT analysis may be used to determine a phase behavior of the reservoir fluid under steady state conditions. That is, conditions for the PVT analysis may be kept constant over at least a period of time during the PVT analysis, and the temperature control fluid is not flowing. The PVT analysis may include a plurality of stages, and a desired temperature for each stage may be different. In a conventional PVT system, for example when an oven is used, a waiting time is required between different stages for temperature equilibrium. As such, there exists a need for a PVT system with a short equilibrium time, and the PVT system described herein realizes the short equilibrium time through a fluid supply unit.

During the PVT analysis, a temperature of the sample cell is controlled by a temperature control fluid. The temperature control fluid may be obtained from the fluid supply unit described in one or more embodiments of the present disclosure, including two or more containers holding fluids at different temperatures. The two or more fluids may include at least a first fluid having a first temperature and a second fluid having a second temperature that is different from the first temperature. The first fluid and the second fluid may be preheated to desired temperatures for different stages of the PVT analysis. For example, in step 203, for a first stage of the PVT analysis, the first fluid having a first temperature may be supplied to the PVT system as the temperature control fluid, the first temperature being the desired temperature for the first stage. In step 204, for a second stage of the PVT analysis, the second fluid having a second temperature, which is different from the first temperature, may be supplied to the PVT system as the temperature control fluid, the second temperature being the desired temperature for the second stage. The heating and supplying of the fluids may be controlled by a controller described in the present disclosure. The temperature control liquid may be introduced to an interior chamber in a temperature control unit of the PVT system, in which the sample cell holding the reservoir fluid to be analyzed is disposed within the interior chamber. In a case when the PVT analysis is performed under steady state conditions, the temperature control fluid may be introduced to the interior chamber, and kept in the interior chamber without disturbance for a period of time, until a thermal equilibrium is established. That is, a temperature in the sample cell is substantially the same as the temperature within the interior chamber, and is substantially the same as the temperature of the temperature control fluid once thermal equilibrium is established.

At steps 203 and 204, the PVT analysis is conducted under the first temperature for the first stage. After the first stage, the second fluid may replace an entirety of the first fluid in the interior chamber, such that a thermal equilibrium of the second temperature immediately established after the replacement, within a short period of time. The PVT analysis may substantially be conducted at the second temperature. The first fluid may be recycled back to the first container. In one or more embodiments, the first fluid in the first container may be preheated to a desired temperature of a third stage of the PVT analysis. Alternatively, the fluid supply unit may include a third container holding a third fluid having a third temperature that is different from the first and the second temperature.

In one or more embodiments, the temperature of the temperature control fluid after thermal equilibrium is established is selected to be a downhole or reservoir temperature from which the reservoir fluid was taken, to simulate a reservoir environment. For example, the reservoir fluid collected from a location in a reservoir under a reservoir pressure and a reservoir temperature (which may be measured using, for example, downhole sensors) may be subjected to PVT analysis under the same reservoir temperature from which the fluid sample was collected.

The method according to one or more embodiments may further include a step 205 of controlling a pressure inside the sample cell. The pressure may be controlled, to increase or to decrease, by a piston, as shown in the non-limiting example of FIG. 1, a pump, or other techniques known in the art. During PVT analysis, the pressure may be kept constant at a certain pressure that is determined by the controller discussed in one or more embodiments.

In one or more embodiments, the method disclosed herein further includes a step 206 of determining a property of the reservoir fluid based on results from the PVT analysis. The property may relate to one or more of formation volume factor, solution gas/oil ratio, gas formation volume factor, compressibility, and specific heat capacity of the reservoir fluid.

In one or more embodiments, the method disclosed herein includes determining a specific heat capacity of the reservoir fluid. For example, the specific heat capacity may be obtained by measuring a temperature change of the temperature control fluid having known mass and specific heat capacity. More specifically, the specific heat capacity of the reservoir fluid may be determined based on Equation (1) represented by:

$${c}_{{P}_{R}} * {m}_{R} * Δ {T}_{R} = {c}_{{P}_{T}} * {m}_{T} * Δ {T}_{T} , \qquad ( 1 )$$

where Cₚ is a specific heat capacity of fluid, m is a mass of fluid, ΔT is a temperature change before and after thermal equilibrium is established, and R and T represents the reservoir fluid and the temperature control fluid, respectively. As an example, a temperature change of the reservoir fluid may refer to a difference between an equilibrium temperature of the reservoir fluid and an initial temperature of the reservoir fluid when introduced to the interior chamber.

In one or more embodiments, a heat loss is taken into consideration when determining the specific heat capacity of the reservoir fluid. The heat loss may be determined based on Equation (2) represented by:

$$q = ( U \times A ) \times Δ T ,$$

where q is a total heat loss, U is an overall coefficient of heat transmission, A is a surface area, and ΔT is a temperature difference between temperatures inside and outside the housing.

In one or more embodiments, a heat transfer to the sample cell through convection is taken into consideration when determining the specific heat capacity of the reservoir fluid. The transferred heat may be determined based on Equation (3) represented by:

$$Q = hA Δ T ,$$

where [symbol] is a heat transferred per unit time, A is an area of the object, and h is a heat transfer coefficient.

In one or more embodiments, a heat transfer to the sample cell through convection is taken into consideration when determining the specific heat capacity of the reservoir fluid. The transferred heat may be determined based on Equation (4) represented by

$${Q}_{w} = - kA Δ T ,$$

where −k is a thermal conductivity of a material composing a wall of the sample cell, A is an area of the object, and h is the heat transfer coefficient.

In one or more embodiments, the method disclosed herein includes determining a property of the reservoir fluid by varying a pressure or a volume of the reservoir fluid under a constant temperature. When the pressure is varied, a change in the volume may be monitored. Similarly, when the volume is changed, a corresponding change in the pressure may be monitored. For example, a bubblepoint may be determined by varying pressure/volume under constant temperature, so as to understand if the reservoir fluid is undersaturated. In one or more embodiments, the method disclosed herein includes determining a property of the reservoir fluid by varying a temperature of the reservoir fluid under a constant volume, and a pressure change may be monitored. In one or more embodiments, the method disclosed herein includes determining a property of the reservoir fluid by varying a temperature of the reservoir fluid under a constant pressure, and a change in volume may be monitored. While limited number of examples are listed in the present disclosure, one having ordinary skill in the art would recognize that PVT analysis performed in conventional PVT systems and methods may also be realized in the PVT system and method disclosure herein, with an enhanced efficiency, especially a shorter time to reach thermal equilibrium, and a reduced volume of device, when the use of oven is avoided.

The property of the reservoir fluid may be determined by a controller described in one or more embodiments. In some embodiments, information related to the property of the reservoir fluid may be transmitted from the one or more components in the PVT system to the controller.

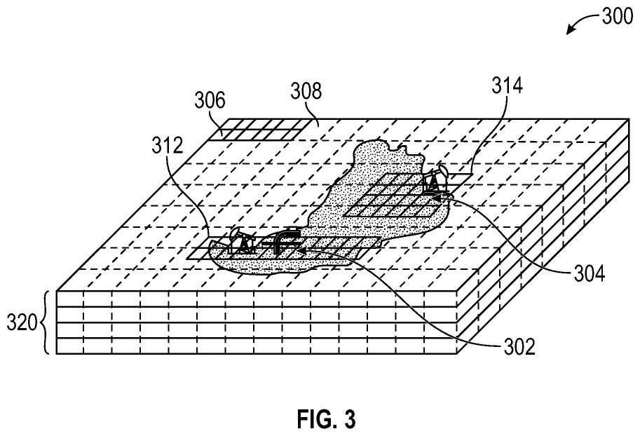

In one or more embodiments, the method disclosed herein further includes a step 207 of generating a reservoir model based on the property of the reservoir fluid. Details regarding the generation of the reservoir model will be discussed in detail, with reference to a non-limiting example of a reservoir simulator shown in FIG. 3.

Reservoir modeling is an important step in the development and production of oil and gas fields. An accurate three-dimensional reservoir model may reduce uncertainties and drilling risks and leads to a more realistic productive forecast. To this end, prediction of reservoir's structural framework together with reservoir properties for both rock formations and fluids are regarded as important steps in reservoir simulation models.

One or more embodiments of the present disclosure relates to a reservoir simulator as shown in FIG. 3. A reservoir grid model 300 may correspond to a geological region that spans a subsurface region of interest. Well sites may be located in the geological region, and may include an injection well 302, which injects a fluid into the local subsurface formations, and/or an extraction well 304. The reservoir grid model 300 may include grid cells 306 that may refer to an original cell of a reservoir grid model as well as coarse grid blocks 308 that may refer to an amalgamation of original cells of the reservoir grid model. For example, a grid cell may be the case of a 1×1 block, where coarse grid blocks may be of sizes 2×2, 4×4, 8×8, etc. Both the grid cells 306 and the coarse grid blocks 308 may correspond to columns for multiple model layers 320 within the reservoir grid model.

Prior to performing a reservoir simulation, local grid refinement and coarsening (LGR) may be used to increase or decrease grid resolution in a certain area of reservoir grid model 300. As shown in FIG. 3, the reservoir grid model 300 may include various fine-grid models (i.e., fine-grid model A 312, fine-grid model B 314), that are surrounded by coarse grid blocks 308. In some embodiments, a reservoir grid model (or multiple reservoir grid models) may be used to perform reservoir simulations.

Generally, reservoir simulators solve a set of mathematical governing equations that represent the physical laws that govern fluid flow in porous, permeable media. In one or more embodiments, the reservoir simulator comprises functionality for simulating the flow of fluids, including hydrocarbon fluids such as oil and gas, through a hydrocarbon reservoir composed of porous, permeable reservoir rocks in response to natural and anthropogenic pressure gradients. The reservoir simulator may be used to predict changes in fluid flow, including fluid flow into well penetrating the reservoir as a result of planned well drilling, and fluid injection and extraction. For example, the reservoir simulator may be used to predict changes in hydrocarbon production rate that would result from the injection of water into the reservoir from wells around the reservoir's periphery. The reservoir simulator may account for, among other things, the porosity and hydrocarbon storage capacity of the subsurface formations and fluid transport pathways to predict the production rate of hydrocarbons of a well, or a set of wells, over their lifetime.

Accurate reservoir models are critical to reduce exploration risks, plan the location of well sites, optimize reservoir production, and better extend hydrocarbon recovery from existing wells. In particular, property characterization of the reservoir fluid as described in the system and method herein may be used to calculate well inflow performance, and to determined fluid flow behavior as the reservoir fluid is surfaced and transported, such as through a pipeline, to processing facilities. Further, the characterization of reservoir fluid properties may be used to determine the requirements of processing facilities.

According to one or more embodiments, some thermophysical properties (e.g., viscosity, specific heat, conductivity) of the reservoir fluid are dependent on pressure and temperature. For downhole applications, evaluation of these thermophysical properties, and other properties of the subsurface (e.g., porosity), may be complicated by the fact that the wellbore temperature and pressure changes substantially from the reservoir to the surface. Thus, fluids that are produced from the reservoir can experience a dramatic change in their thermophysical properties as they are brought to the surface. It is noted that reservoir fluid often undergoes a phase change both in the subsurface and when the fluids are surfaced. Accordingly, the systems and methods disclosed herein provide strategies for fast PVT analysis to understand phase behavior or other properties of reservoir fluids and may facilitate the oilfield application in various aspects discussed above.

In one or more embodiments, the reservoir modeling and simulating and/or prementioned methods and systems disclosed herein may be performed by a computer system shown in FIG. 4. Specifically, FIG. 4 depicts a block diagram of a computer system 400 used to provide computational functionalities associated with the methods and procedures as described in this disclosure, according to one or more embodiments (e.g., the controller 150). The illustrated computer 402 is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer 402 may include a computer that includes an input device, such as a keypad, keyboard, touch screen, or other device that can accept user information, and an output device that conveys information associated with the operation of the computer 402, including digital data, visual, or audio information (or a combination of information), or a Graphical User Interface (GUI).

The computer 402 can serve in a role as a client, network component, a server, a database or other persistency, or any other component (or a combination of roles) of a computer system for performing the subject matter described in the instant disclosure. The illustrated computer 402 is communicably coupled with a network 430. In some implementations, one or more components of the computer 402 may be configured to operate within environments, including cloud-computing-based, local, global, or other environment (or a combination of environments).

At a high level, the computer 402 is an electronic computing device operable to receive, transmit, process, store, or manage data and information associated with the described subject matter. According to some implementations, the computer 402 may also include or be communicably coupled with an application server, e-mail server, web server, caching server, streaming data server, business intelligence (BI) server, or other server (or a combination of servers).

The computer 402 can receive requests over network 430 from a client application (for example, executing on another computer 402) and responding to the received requests by processing the said requests in an appropriate software application. In addition, requests may also be sent to the computer 402 from internal users (for example, from a command console or by other appropriate access method), external or third-parties, other automated applications, as well as any other appropriate entities, individuals, systems, or computers.

Each of the components of the computer 402 can communicate using a system bus 403. In some implementations, any or all of the components of the computer 402, both hardware or software (or a combination of hardware and software), may interface with each other or the interface 404 (or a combination of both) over the system bus 403 using an application programming interface (API) 412 or a service layer 413 (or a combination of the API 412 and service layer 413. The API 412 may include specifications for routines, data structures, and object classes. The API 412 may be either computer-language independent or dependent and refer to a complete interface, a single function, or even a set of APIs. The service layer 413 provides software services to the computer 402 or other components (whether or not illustrated) that are communicably coupled to the computer 402. The functionality of the computer 402 may be accessible for all service consumers using this service layer. Software services, such as those provided by the service layer 413, provide reusable, defined business functionalities through a defined interface. For example, the interface may be software written in JAVA, C++, or other suitable language providing data in extensible markup language (XML) format or another suitable format. While illustrated as an integrated component of the computer 402, alternative implementations may illustrate the API 412 or the service layer 413 as stand-alone components in relation to other components of the computer 402 or other components (whether or not illustrated) that are communicably coupled to the computer 402. Moreover, any or all parts of the API 412 or the service layer 413 may be implemented as child or sub-modules of another software module, enterprise application, or hardware module without departing from the scope of this disclosure.

The computer 402 includes an interface 404. Although illustrated as a single interface 404 in FIG. 4, two or more interfaces 404 may be used according to particular needs, desires, or particular implementations of the computer 402. The interface 404 is used by the computer 402 for communicating with other systems in a distributed environment that are connected to the network 430. Generally, the interface 404 includes logic encoded in software or hardware (or a combination of software and hardware) and operable to communicate with the network 430. More specifically, the interface 404 may include software supporting one or more communication protocols associated with communications such that the network 430 or interface's hardware is operable to communicate physical signals within and outside of the illustrated computer 402.

The computer 402 includes at least one computer processor 405. Although illustrated as a single computer processor 405 in FIG. 4, two or more processors may be used according to particular needs, desires, or particular implementations of the computer 402. Generally, the computer processor 405 executes instructions and manipulates data to perform the operations of the computer 402 and any algorithms, methods, functions, processes, flows, and procedures as described in the instant disclosure.

The computer 402 also includes a memory 406 that holds data for the computer 402 or other components, such as computer executable instructions, (or a combination of both) that can be connected to the network 430. The memory 406 may be non-transitory computer readable memory. For example, memory 406 can be a database storing data consistent with this disclosure. Although illustrated as a single memory 406 in FIG. 4, two or more memories may be used according to particular needs, desires, or particular implementations of the computer 402 and the described functionality. While memory 406 is illustrated as an integral component of the computer 402, in alternative implementations, memory 406 can be external to the computer 402.

The application 407 is an algorithmic software engine providing functionality according to particular needs, desires, or particular implementations of the computer 402, particularly with respect to functionality described in this disclosure. For example, application 407 can serve as one or more components, modules, applications, etc. Further, although illustrated as a single application 407, the application 407 may be implemented as multiple applications 407 on the computer 402. In addition, although illustrated as integral to the computer 402, in alternative implementations, the application 407 can be external to the computer 402.

There may be any number of computers 402 associated with, or external to, a computer system containing computer 402, wherein each computer 402 communicates over network 430. Further, the term “client,” “user,” and other appropriate terminology may be used interchangeably as appropriate without departing from the scope of this disclosure. Moreover, this disclosure contemplates that many users may use one computer 402, or that one user may use multiple computers 402.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A pressure-volume-temperature (PVT) system for characterization of a reservoir fluid, comprising:
a temperature control unit;
a sample cell disposed inside the temperature control unit for accommodating the reservoir fluid,
wherein the temperature control unit comprises a housing and an interior chamber defined by a space between the sample cell and the housing; and
a fluid supply unit that provides a temperature control fluid to the interior chamber to control a temperature of the sample cell;
wherein the fluid supply unit includes a first container providing a first fluid having a first temperature as the temperature control fluid for a first stage of PVT analysis and a second fluid having a second temperature that is different from the first fluid as the temperature control fluid for a second stage for PVT analysis.

2. The PVT system of claim 1, further comprising a piston arranged in the sample cell and configured to control a pressure inside the sample cell.

3. The PVT system of claim 1, wherein the first container is configured to preheat the first fluid to the first temperature before the first stage and preheat the second fluid to the second temperature before the second stage.

4. The PVT system of claim 1, wherein the second fluid replaces the first fluid in the interior chamber after the first stage, such that a temperature in the interior chamber changes from the first temperature to the second temperature after replacement.

5. The PVT system of claim 1, wherein the reservoir fluid is characterized in the sample cell under steady state conditions to obtain a property of the reservoir fluid.

6. The PVT system of claim 5, further comprising a controller that processes data obtained from the sample cell to determine the property of the reservoir fluid for use in a reservoir model.

7. A method for performing a pressure-volume-temperature (PVT) analysis, comprising:
collecting a reservoir fluid from a reservoir;
performing PVT analysis on the reservoir fluid using a sample cell disposed in a temperature control unit;
controlling a temperature of the sample cell using a temperature control fluid in an interior chamber defined by a space between the sample cell and a housing of the temperature control unit,
providing, for a first stage of the PVT analysis, a first fluid having a first temperature as the temperature control fluid, and
providing, for a second stage of the PVT analysis, a second fluid having a second temperature that is different from the first temperature as the temperature control fluid.

8. The method of claim 7, further comprising controlling a pressure inside the sample cell by a piston arranged in the sample cell.

9. The method of claim 7, further comprising preheating the first fluid in a first container to the first temperature before the first stage, and preheating the second fluid in a second container to the second temperature before the second stage.

10. The method of claim 7, further comprising replacing the first fluid in the interior chamber with the second fluid after the first stage, such that a temperature in the interior chamber changes from the first temperature to the second temperature after replacement.

11. The method of claim 7, further comprising determining a property of the reservoir fluid by characterizing the reservoir fluid in the sample cell under steady state conditions.

12. The method of claim 11, further comprising processing data obtained from the sample cell to determine the property of the reservoir fluid for use in a reservoir model.
